(19) United States
(12) Patent Application Publication
(10) Pub. No.: US 2011/0213197 A1
(43) Pub. Date: Sep. 1, 2011

(54) Computer augmented therapy

(76) Inventors: Bruce D. Robertson, Poulsbo, WA (US); Bernard Robert Franza, Seattle, WA (US)

(21) Appl. No.: 12/932,603

(22) Filed: Feb. 28, 2011

Related U.S. Application Data
(60) Provisional application No. 61/308,595, filed on Feb. 26, 2010.

(51) Int. Cl. A61M 21/00

(52) U.S. Cl. 600/27

(57) ABSTRACT

A method to treat sympathetic dysfunction using computer-augmented therapy, such as computer augmented imagery rehearsal therapy, comprising the steps of (a) achieving a relaxed state calibrated by ART; (b) accessing a machinima using a virtual reality system; (c) creating an avatar in said machinima; (d) developing a script for a virtual scenario; (d) creating said virtual scenario in said machinima; (e) reinforcing virtual scenario in said machinima through repetitive practice of said dream scenario and (f) practicing said virtual scenario during a dysfunction episode to achieve control over sympathetic arousal.

### CROSS-REFERENCE TO RELATED APPLICATION

This application claims priority to U.S. provisional application 61/308,595 filed Feb. 26, 2010.

### FIELD OF INVENTION

This invention relates to a computer augmented (computer-based) neuro-physiological training model for self-regulating sympathetic dysfunction. In particular, this invention relates to methods for treating nightmares and acute pain episodes using imagery rehearsal techniques in a virtual reality environment. The invention can be used in medical, psychiatry, psychotherapy, education, self-help, and home settings with appropriate computer hardware and software.

### BACKGROUND

Stress plays an important role in mental disorders, as both a causal factor and an outcome of disordered thought and disrupted interpersonal relationships. Events that are threatening to an individual and that elicit physiological and behavioral responses are defined as stressors. They include events that occur routinely during the course of the day, as well as non-routine physically and/or psychologically health-threatening events.

There are two key aspects of the body's response to stressors. On one hand, the body responds to many stressors by releasing chemical mediators, such as catecholamines that increase heart rate and blood pressure. These mediators promote adaptation to an acute stressor, as well as to simple acts like getting out of bed. On the other hand, chronic elevation of these same mediators, e.g. chronic increased heart rate and blood pressure, can lead to pathophysiological changes [19].

The term “allostasis” (stability through change) is a term introduced by Sterming and Eyer, and refers to the active process by which the body responds to daily events and maintains homeostasis. Allostasis achieves adaptative adjustment to stressors by resetting target values for physiological variables, a different way of sustaining system viability than Bernard-Cannon model of homeostasis, which attempts to re-achieve a steady-state with pre-stressed values for physiological variables.

Allostatic load refers to the long-term result of failed adaptation or failed allostasis, resulting in pathology and chronic illness [19]. FIG. 1, shows four types of allostatic load. The top panel illustrates normal allostatic response, in which a response is initiated by a stressor, sustained for an appropriate interval, and then turned off. The remaining panels illustrate four conditions that lead to allostatic load. The top left panel shows repeated “hits” from multiple stressors, which is the case of simply too much “stress” in the form of repeated, novel events that cause repeated elevations of stress mediators over long periods of time. The top right panel illustrates a failure to habituate or adapt to the same stressor. This leads to the over-exposure to stress mediators because of the failure of the body to dampen or eliminate the hormonal stress response to a repeated event. An example of this is the finding that after repeated public speaking challenges; a significant minority of individuals fails to habituated and continue to show cortisol response (14). The bottom left panel shows prolonged response due to delayed shut down by the body. One example of this is blood pressure elevations in work-related stress, which turn off slowly in some individuals with a family history of hypertension [11]. Another example is that of sleep deprivation leading to elevated evening cortisol and hyperglycemia within 5d and depressive illness leading to chronically elevated cortisol and loss of bone mineral mass. The bottom right panel illustrates inadequate response that leads to compensatory hyperactivity of other mediators [19]. For example, an inadequate hormonal stress response, which allows other systems, such as the inflammatory cytokines, to become overactive. The Lewis rat is an example of increased susceptibility to inflammatory and autoimmune disturbances is related to inadequate levels of cortisol.

The relationship between psychological stresses has been linked with some physiological disorders. For example, psychological distresses are found to associate with symptoms of idiopathic pain disorders, such as irritable bowel syndrome and fibromyalgia, and temperomandibular joint disorder [19]. In another example, mental stress has also been associated with sleep deprivation and increased food intake.

Neuroplasticity (also known as cortical re-mapping) refers to the ability of the human brain to change as a result of one's experience. The brain consists of nerve cells (or “neurons”) and glial cells which are interconnected, and learning may happen through change in the strength of the connections, by adding or removing connections, and by the formation of new cells.

Problematic behaviors, such as nightmares, and anxiety pursuant to these negative dream states are programmed through repetitive experience. In order to override these, a neurological state in which novel (new) learning can occur must be created. The new state or a state of receptivity can be created via attentive relaxation. The therapeutic goal then becomes to fire and wire new neurological networks by programming the brain with visual and auditory stimuli that trigger parasympathetic pathways to counter the arousal (sympathetic) networks.

Imagery Rehearsal Therapy (IRT) is a treatment developed by Barry Krakow and colleagues as an efficacious treatment for patients with chronic nightmares [17]. In 2001, Krakow et al. reported the significant improvement of Sleep Quality and a reduction of PTSD symptoms among sexual assault survivor who are treated with imagery rehearsal therapy for chronic nightmares. Krakow, et al. have also studied the effectiveness of imagery rehearsal therapy on acute PTSD among Iraq combat soldiers and saw a 61% reduction in nightmares, a 41% reduction in PTSD symptoms within 1 months of the treatment and 34% reduction in chronic insomnia [18].

The imagery rehearsal treatment is delivered in five 60-minute weekly sessions provides patients with psycho-education on sleep, nightmares, imagery rehearsal. Patients with recurring nightmares are asked to write out or talk about the text of the nightmare in as much detail as they can remember no matter how frightening. They are also required to track nightmares nightly for both frequency and intensity. Patients are then taught the skill of guided pleasant imagery using standard images, and are then asked to develop their own personalized pleasant imagery scene that they practice for at least ten minutes twice daily. Patients are then asked to select one target nightmare to “rescript” in which the therapist teaches patients to identify and elaborate upon an alternative, neutral and/or pleasant ending for the target nightmare. Again, an imagery rehearsal schedule is established and patients are encouraged to rehearse the “new dream” imagery each night just before going to sleep as they did the guided pleasant imagery. Progress is then reviewed, problems are addressed, and then relapse prevention is emphasized. Since it is believed that the nightmare is grounded in emotions, such as raw anger, that have been provoked by a trauma, the aim of this step is to “tame” the emotions, not merely vent them in violence and revenge.

Image rehearsal therapy differs from traditional CBT such as exposure therapy by focusing on the anxiety response related to nightmares rather than the stressor. In exposure therapy, patient is trained to build tolerance via exposure to stressor which caused the psychological response. In IRT, the therapist provides the user with tools to imagine more secure imagery, and thus diminish the negative emotions.

In order to be successful in IRT, patients must have effective imagery skills. For some patients, imagery is challenging and they have insufficient imagery skills to produce a change in behavioral conditioning. If patients do not have sufficient imagery skills, then IRT is unlikely to work as it is currently designed. Among the various existing technologies, virtual reality, have the potential to be effective in assisting those who have difficulty with imagery develop the necessary skill.

The term “virtual reality” has been used to describe a computer-generated environment. When viewed with goggles or head-mounted display, it provides the user with a three-dimensional, fully interactive experience. A hand-held grip may be used to achieve movement or navigation within the environment. As the user turns his or her head, the view changes just as it would in reality. Buttons on the hand-held grip permit the user to experience movement from one location to another, thus adding a sense of reality, to virtual reality environment. The technology used to produce virtual reality may include a graphics-generating computer, a head-mounted-display with a tracking device, a hand-held grip, and other sensory input devices. Various products may be used to achieve the experience of virtual reality [1]. Virtual reality applications have been developed for art, business, entertainment, flight simulators, medicine, and military battlefield operations. Medical applications included computer-aided surgery, building designs for handicapped persons, wheelchair equipped with a virtual reality system, rehabilitation, repetitive strain injury, surgical workstation, and teaching aids for surgeons.

Clinicians have attempted to incorporate virtual reality applications into treatment of certain psychological conditions using exposure therapy. An apparatus and method for treating undesirable emotional arousal of a user is shown by Weathers in U.S. Pat. No. 5,219,322 (1993). This invention uses visual and auditory stimuli as a crude process for eliciting mental imagery of a negative experience. The primary goal of psychotherapy is to provide corrective experiences that can be effectively used by patients. The more closely the corrective experience simulates reality, the more effective the treatment. However, Weathers does not use any fully interactive visual and auditory stimulation that are under the control of the patient. It does not permit the user to influence the environment as well as be influenced by it.

Lamson in U.S. Pat. No. 6,425,764 (1993) suggested the use of an immersive, 3D, fully interactive virtual reality technology as part of cognitive behavioral therapy (CBT) for the experimental treatment of acrophobia, anxiety disorders, mood disorders and substance-related disorders. CBT is used in individual therapy as well as group settings, and the techniques are often adapted for self-help applications. The method described in Lamson seeks to provide the patients with assessment of cognitive, emotional, and physiological functioning. It is also used for prevention and treatment of psychiatric conditions by providing users corrective experiences through counseling and biofeedback such as exposure therapy.

Carlin et al. (1997) present a case report to demonstrate another application of virtual reality exposure therapy. Immersive computer generated virtual reality (VR) and mixed reality (touching real objects seen in virtual reality) was used for the treatment of spider phobia. A user was exposed to virtual spider scenes over 12 weeks with each session lasting a total of 50 minutes. Exposure to virtual reality spiders produced reduction in anxiety with some symptom relief.

In addition to exposure therapy, many other applications of computer augmented reality have been explored. Gould, in U.S. Pat. No. 5,546,943 (1996) proposes use of a visualization system using a computer to provide a user with a view of their internal anatomy based on medical scan data. The user acts upon the information in an interactive virtual reality environment by using tools or other devices to diminish a visual representation of an ailment. In doing so, a psychoneuro-immunological response is postulated to occur in the user for combating and recovering from the disease, which may be due to any process that builds an individual's self-efficacy.

Jarvik, in U.S. Pat. No. 5,577,981 (1996) describes a virtual reality exercise machine and computer controlled video system. Jarvik's machine produces a virtual reality environment for exercise regimens, exercise games, competitive sports, and team sports. It is used to achieve exercise results from rehearsal and can be adapted to a user's individual capabilities.

Walker, Lyon, Linton, and Nye, in U.S. Pat. No. 5,584,696 (1996) describes a simulation system for virtual reality experiences such as hang gliding or the like. They describe an embodiment for mechanical support, visual display, and a method for achieving pupil-forming images.

Other than clinical setting, virtual realities have also been explored in education and self-help products. U.S. Pat. No. 4,573,472 (1986) by Ito shows a medical apparatus for autogenic training. The self-help training procedure operates by providing bio-information stimuli. The user is expected to consider that information and alter behavior. However, this training apparatus does not provide sensory stimulations that evoke thinking distortions (fear), anticipatory anxiety, danger expectations, failure beliefs, physiological reactions (anxiety, deep breathing or holding of breadth, sweating) during exposure. The lack of visual exposure, auditory and tactile stimulations does not permit the practitioner to immediately introduce interventions for the purpose of achieving corrective experience. Variables that influence behavior, such as self-efficacy, cannot be assessed and strengthened during immersion of the user in a virtual environment.

Densky, in U.S. Pat. No. 4,717,343 (1988) shows a method for conditioning a person's unconscious mind to effect a desired change in behavior. This self-help method exposes a person to a video picture appearing on a screen. The procedure claims that the viewer's unconscious mind observes the video and that somehow the viewing conditions a person's thought patterns that alter behavior in a positive way. However, the procedure does not use known learning principles and sensory stimulations is not fully-immersive.

Today, there is not a system allowing full immersion of a patient in a virtual reality system, which can be modified by the patient to achieve positive self-conditioning. This invention seeks to combine the immersion power of virtual reality, with IRT to increase the sense of reality for the patient, thus increase the power of the imagery and the effectiveness of the treatment.

### BRIEF DESCRIPTION OF FIGURES

FIG. 1. Shows normal allostasis and allostatic load.

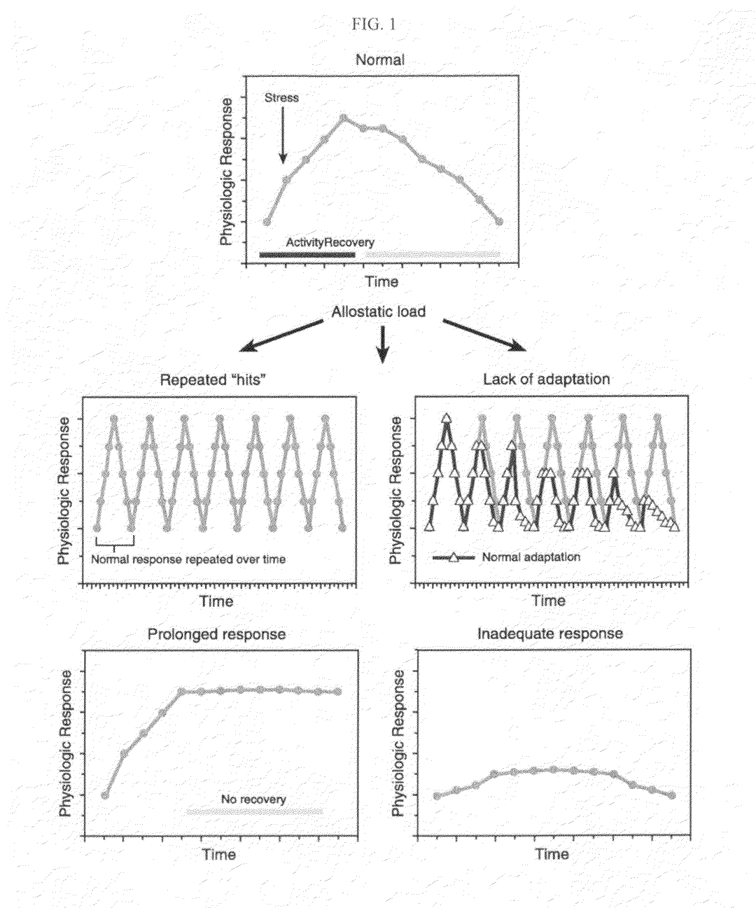

FIG. 2 is block diagram of an embodiment of the system supporting computer-augmented therapy.

FIG. 3 is a second flowchart illustrating the use of computer-augmented therapy for chronic nightmare.

FIG. 4 is a third flowchart showing the use of computer-augmented therapy for pain management.

FIG. 5 illustration of an exemplary dream scenario.

### DETAILED DESCRIPTION OF INVENTION

Computer-augmented therapy of this invention is an application of Neuro-Physiological Self Regulation Training (NPSRT). NPSRT is the integration of Autonomic Regulation Training (ART) and Virtual Reality Training (VRT). Autonomic regulation training (ART) is simply programming the mind and body to develop an optimally calm (parasympathetic) state for learning. This is well known in the art, physiological parameters such as heart rhythms are inherently connected to brain wave activity. When the variability of the heart is under control by increasing tidal volume of oxygen and decreasing the rate of respirations, the brain adjusts by increasing the predominance of alpha waves. The heart rate variability biofeedback training has been utilized in clinical practice for over thirty years to address a wide range of disorders. By having these changes feedback visually via the computer the user learns to control his/her heart coherence, and achieve the desired receptive and calm state. In an embodiment of the ART, a user can use surface EMG electrodes placed on the wrist to generate a heart signal, which is portrayed on the computer monitor in various graphic representations.

Virtual Reality Training (VRT) is simply using computer based visual and auditory stimuli to provide the input for new therapeutic learning. VRT can be done by developing software within an existing virtual environment (such as Second Life™), providing machine animation tools so the subject to create images and sounds that trigger the parasympathetic process. With repetition the user learns to control the arousal associated with the disorder. In an embodiment of the VRT, the subject logs into the identified virtual world and accesses the software through a specific portal available only to selected users. The user utilizes animation tools and icons to construct 3D animation customized to fit the imagination of the user. In both ART and VRT, the therapeutic process is augmenting with technology.

Visual, auditory and tactile sensory stimulation during user immersion in virtual reality are used to assist the user in achieving corrective experiences. The instillation of explicit learning principles before and during virtual environment exposure permit user to directly influence psychological, emotional, and physiological processes for the development of mental health. The computer-augmented therapy of this invention differs from prior art in that it is not a form of exposure therapy. The method does not require repeatedly challenging the user with stressful experiences in hope of building tolerance to the stressor. The method works by “augmenting” the ability of the user to develop vivid imagery that are used to override the acute arousal associated with the stressor to achieve parasympathetic stasis. The user use tools provided by the augmented virtuality system to create imageries unique to his/her neurology. Possible application of this therapy may include insomnia, acute pain, rage, panic attacks or any other reactive human process.

The computer-augmented therapy of this invention primarily take place during immersion of fully interactive three-dimensional virtual reality environment, which is supported by a virtual reality system build with computer generated graphics, images imported from photographs, and video for sensory stimulation. A good example of such a virtual reality system is computer simulations such as Second Life™ or similar simulations that present a virtual world which allows users or players to be represented by characters known as avatars. Second Life is an Internet-based virtual world launched in 2003 by Linden Research, Inc. A downloadable client program called the Second Life Viewer enables users, called “Residents”, to interact with others in the virtual world through motional avatars. The virtual world basically simulates the real world or environment. The users or residents via their avatar can explore the virtual world, meet other users or residents, socialize, participate in individual and group activities, and/or create and trade items (virtual property) and services from one another.

In a general embodiment of the inventive method, interactive immersion of the patient is achieved with 3-D goggles, head-mounted display or another form of visual display, such as computer monitors, TV screens or other devices that permit the user to have a virtual experience. The interactive environment permits the use of device such as stereo earphones to receive auditory cues, such as voice, music, natural sounds. Body sensors and devices such as a hand-held grip, or computer mouse, are used to permit the user to interact with virtual objects and navigate within the virtual environment.

Additionally, the user may create avatars using a technology similar to those provided by evolver.com, which allows individuals to upload a single photo to create an avatar “clone” and to create custom avatars and avatar clothing. Users have the capability to customize their avatars themselves throughout their use of the virtual reality system. All the functionality for creating user profiles exists in the virtual reality environment, but a small amount of software development work is needed to create a user interface to this functionality. It would be possible to pull in user profile information from an identity management system, including LDAP, or from a social networking tool.

The themes of the virtual reality environments are generally limited only by the ability of the administrator and developer to create a scenario, and then script, build and run it on the available hardware platform. The user is able to utilize a customizable backdrop provided by a selected theme as a context in which their personal imagery can be generated. These environments are built on objects created with primitive objects and activated by proprietary scripting languages. Other virtual reality environments are created using 3D models stored in a variety of formats. Almost all commercial and open source 3D modeling tools such as SketchUp, Blender, Maya, or 3D Studio Max can export to the Collada format used by most virtual reality systems. Assets found in Google 3D Warehouse and Google Earth can also be imported to these virtual reality environments. Most of these models, which are freely available, can be dragged and dropped into the virtual reality environment, and then positioned or resized using the editing tools within the virtual reality system. All the models can also be defined to include object-level security, restricting access to appropriate personnel or roles.

In these virtual environments it is also possible to create any interactive scenario with a combination of live and automated characters. For example, the in-world webcam viewer could be used to include a “window” into either a real-life traumatic or peaceful scenario, allowing participants to monitor activity in the real world as well as in the virtual reality environment. In the inventive treatment method, this capability would effectively allow the user to generate customized imagery to recreate the personal experience with expressive details in the virtual environment to neurologically strengthen parasympathetic imagery to counter the dysfunctional pathways created by the trauma exposure.

Physiological parameters of the patient, such as heart rate, blood pressure, respiration and temperature, are measured using one or more monitoring device. An example of a compact monitoring device is described in Appendix A of this application. Users are taught in auto regulation training, which helps them priming their body for effective learning using biofeedback responses measured.

The computer-augmented therapy provides opportunities for self-help when the user of a virtual environment is provided information on how to benefit from the experience or when the provider gives directions on how to benefit from the experience or when the virtual environment itself provides the user with directions on effective user of learning strategies before immersion in the virtual environment.

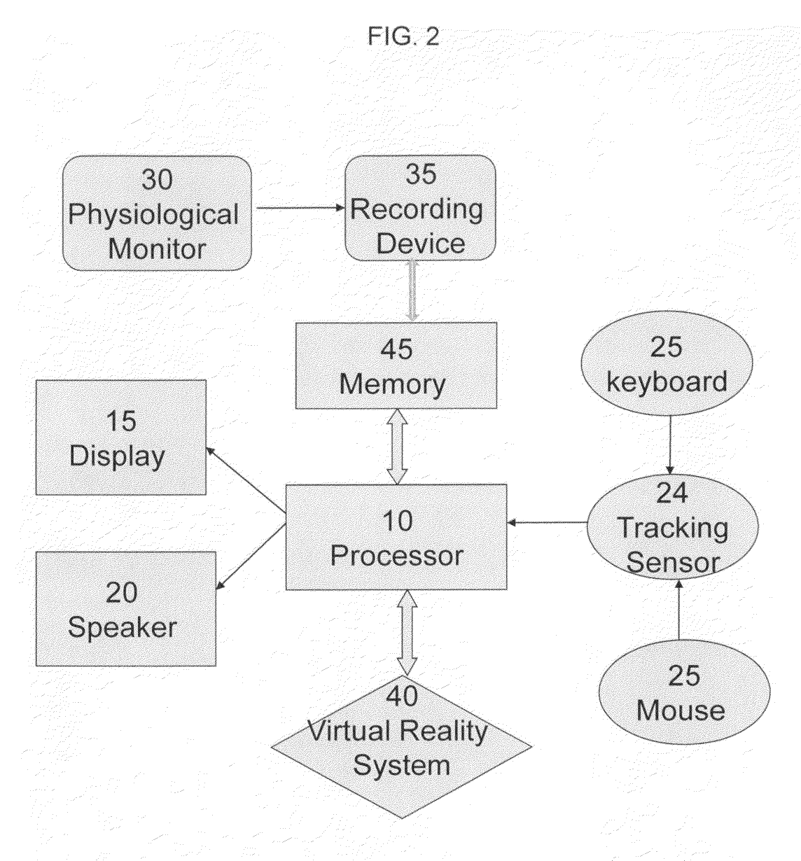

FIG. 2 shows a block diagram of an embodiment of the virtual reality components supporting the inventive therapy method. At the heart of the system is a processor 10, which may be housed in a computer or as part of an independent device. The processor 10 may contain the software program for generating the virtual environment including a depository of virtual imagery, videos or mixed images. Alternatively, the processor may contain software supporting user interface, allowing the user to access a virtual reality system hosted on a remote device, which contains the AR software 40 as well as the image depository required for generating the virtual environment. The processor 10 also relays information between the trackers 25 and the virtual reality system 40. The connection may be between the processor and the remote device may be wireless, radio, or physical. Virtual reality system software 40 provides tools enabling the user to build or accessing his/her personalized virtual environments and may provide the patient with educational instructions. A display 15, which may include a monitor, a head-mounted display, or a 3-D google, is connected to the processor providing the user with visual stimulations and visual instructions. An audio speaker 20, such as a head phone, is connected to the processor to provide the user with auditory stimulations and audio instructions. Various forms of user input devices 25 may also be connected to processor allowing the user to navigate within the virtual environment and interact with the virtual reality system 40. Examples of such input devices include but not limited to a keyboard, mouse, hand-held grip, joystick, triggers and buttons. Tracking sensors 24 receives and sends position location data to the processor, which may in turn communicates with the virtual reality system 40 relaying data bidirectionally. A memory device 45 may also be in communication with the processor 10, allowing data relating to each treatment be recorded, such as the virtual environment build by the users and instructions they received. A physiological monitor 30 is included to measure physiological parameters of the user, which may be recorded by a recording device 35 connected to the physiological monitor. The recording device 35 may be in communication with the memory 45, allowing physical parameters of the user to be stored. Memory 45 can be a hard disk, floppy disk, compact disk (CD), a cartridge, a network storage unit, or any other standard medium capable of storing electronic instructions for running fully interactive, immersive, three-dimensional graphics and storing all data related to the treatment. The ability to hold a large amount of data is a prerequisite for storing large graphic programs.

### General Operation

Before using the virtual reality-based technology shown in FIG. 2, a patient must first visit a health care professional to evaluate his or her psychiatric or medical condition. The practitioner will diagnose the condition and choose the proper treatment plan based on the patient's needs. TABLE 1. Shows the basic processes of virtual therapy, which the mental health professional can use for evaluation and treatment of the user's behavior, medical or emotional conditions such as insomnia or chronic pain.

TABLE 1
Basic Processes of computer-assisted augmented Therapy

1) Develop Practitioner-Patient Relationship
2) Identify presenting problem(s)
3) Obtain history of presenting problem(s)
4) Identify target problems
5) Agree on commitment to tasks leading to successful achievement
of treatment plan
6) Educate the patient about the target problem
7) Screen the patient for computer-assisted augmented training
8) Didactic training on neuroplasticity/allostasis
9) Discuss principles of Neuro-Physiological Self Regulation Training and
learning in immersive VR environment
10) Provide user with HRV biofeedback training/Automatic regulation
training
11) Provide user Virtual Reality Training
12) Home Automatic regulation training
13) Home computer-assisted augmented therapy with immersion in virtual
environment specific to patient presenting complaint(s)
14) Case management oversight and follow-up visit as part of treatment
strategy
15) Post training assessment

### Embodiment 1

### Chronic Nightmare

Nightmares are highly prevalent among patients diagnosed with posttraumatic stress disorder (PTSD) and are among the most frequently reported chronic symptoms of PTSD. Nightmares often persist following standard treatments for PTSD. Recent estimates indicate 50-70% of patients diagnosed with PTSD report frequent nightmares although estimates are higher (90%) in some studies of veterans, with some evidence suggesting that the severity and frequency of the nightmares is associated with the degree of combat and trauma exposure. Although rates are yet to be officially determined for OEF/OIF, early evidence suggests the rate of trauma-related nightmares will be similar in this generation as in previous cohorts. Chronic nightmares are associated with delayed sleep onset, sleep fragmentation, poor sleep quality, and often a conditioned fear of sleep. In addition to nightmares, insomnia is a common complaint in PTSD. Data from the National Vietnam Veterans Readjustment (NVVR) study revealed combat veterans with PTSD were eight times more likely to report sleep onset difficulties than combat veterans without PTSD. In the same study, a staggering 90.7% of veterans with PTSD reported difficulty staying asleep “sometimes” or more frequently, compared to 62.5% of non-PTSD combat veterans and 52.9% of civilian. Fragmented sleep in PTSD is also characterized by atypical, sometimes violent, motor behaviors during sleep, although this may be related more directly to nightmares.

Much evidence suggests insomnia and nightmares become distinct and co-occurring syndromes during the course of PTSD due to ineffective coping strategies to deal with these conditions implemented by patients. In a recent study of Vietnam combat veterans, 88% reported trauma-related nightmares upon entering a three month PTSD treatment program—following the program 77% continued to experience frequent and distressing nightmares. This may not be surprising, given trauma-focused therapies (e.g., Prolonged Exposure) do not involve any sleep or nightmare related treatment techniques, leaving nightmares unaddressed, especially when they have become distinct diagnoses.

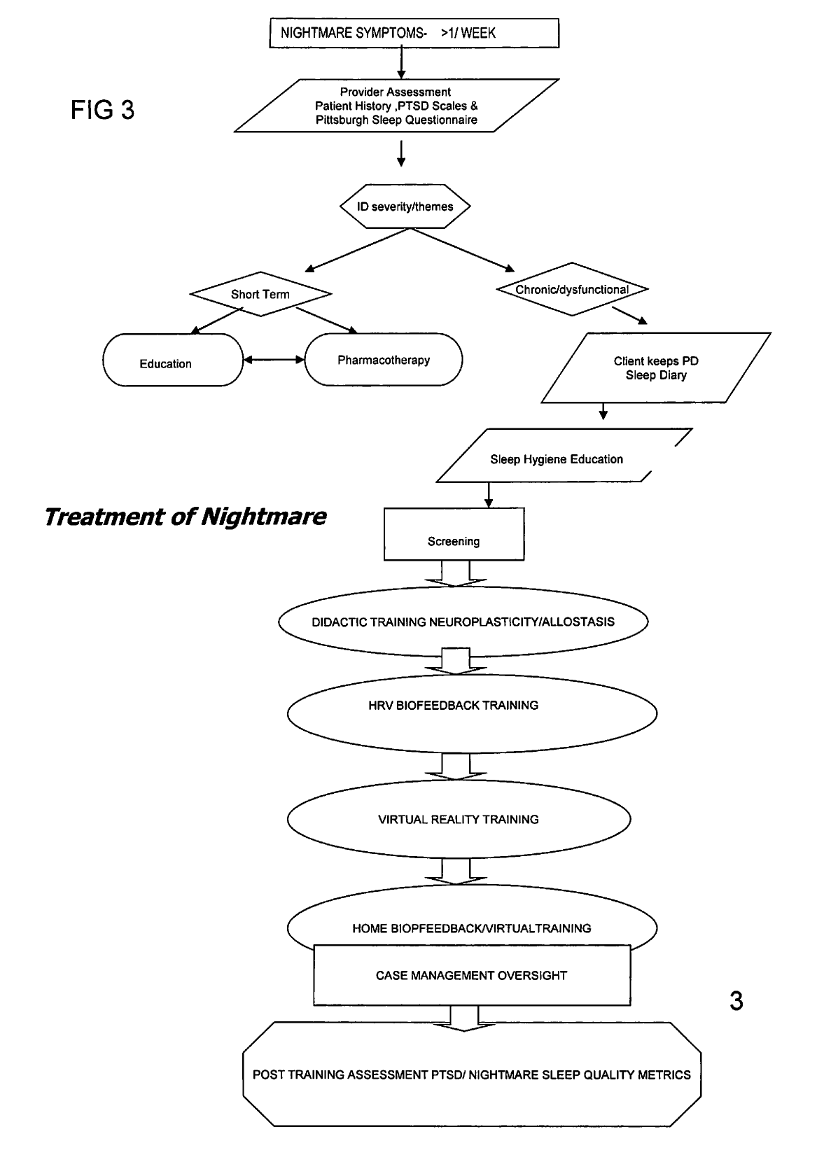

FIG. 3 shows the process of treating a patient with sleep disorder. The process begins with patient assessment/history. The psychological strategies listed include explicit identification of learning principles, cognitive re-framing of distorted thinking processes, and replacement of failure beliefs with success experiences achieved in the virtual environment. The computer-augmented imagery rehearsal therapy works to distract the patient's attention away from sleep-interfering cues such as fear cue from a nightmare. The user is given time and instruction on how to create a prerequisite allostatic parasympathetic stasis via autonomic regulation training. The user is then provided with tools to create personalized relaxation immersive virtual experience. When sensory stimulation impacts vision, hearing, and touch, the user develops adaptive personalized dream scenarios. With each practice and rehearsal, the adaptive personalized dream scenarios are refined and strengthened through the cognitive mediator of self-efficacy.

As shown in FIG. 3, the therapist first works to establish a Practitioner-Patient Relationship via patient interview. The patient is assessed using standard sleep assessment tests, including but not limited to Pittsburg Sleep Quality Index and PTSD Symptom Scales. Based on patient history and assessment, the therapist identifies the severity and diagnoses the patient's sleep disorder. For short term sleep disorder, the therapist may elect traditional therapeutic strategy including sleep education and pharmacotherapy. For patients with chronic/dysfunctional sleep disorders, the therapist may proceed to computer-augmented imagery rehearsal therapy, which begins with sleep hygiene education. Patients are educated on how nightmares are developed based on neuophysiological/physiological models. They are introduced to the theory of neuroplasticity and allostasis, and are taught the roles of automatic (biofeedback) regulation training in priming body to learning and Virtual Reality training in treatment of sleep disorder. An example of sleep hygiene education procedure is provided in Appendix B. After the initial training, patient is then screened for suitability/commitment for the computer augmented imagery rehearsal therapy. Trainee expectation and trainer's role are discussed. Automatic regulation training (ART) is provided to patients elected for computer-augmented imagery rehearsal therapy. The patients are provided with hands on experience using the physiological monitor(s) and are taught on how to use biofeedback to reach allostasis. Patients are given opportunities to practice and familiarize themselves with the physiological monitor(s) under the supervision of a trainer and their physiological baseline metrics are established.

Following the automatic regulation training, patients undergo virtual reality training (VRT). The trainer shows the patient on how to use the virtual therapy software and explains how it works. Patients have hand-on experience with immersive environment, animation, and undergo self-paced tutorials. Patients are then given the opportunity to create his/her own avatar (virtual personality), and their own dream storyboard by using imageries and tools provided by the virtual reality system. The scripts are then powered into the virtual environment and patients are allowed to practice within the virtual environment.

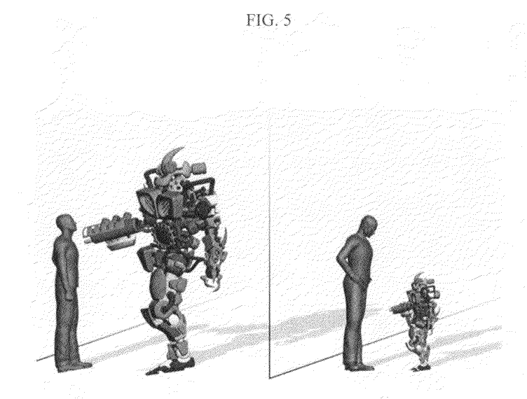

Following ART and VRT trainings, qualified patients are provided with equipments required for the augmented imagery rehearsal therapy, and proceed to perform therapeutic training at home. Patients must complete at home training sessions according to a pre-determined treatment plan. Each treatment session starts with the ART. Patient practices biofeedback relaxation to prepare for VRT learning. Their physiological parameters are measured using the physiological monitor(s). During the follow-up VRT session, patient log onto the virtual reality system and using the imageries and tools provided by the system to build a personalized dream scenario and creating their own machinima (a virtual environment or a animated virtual world). Trainer can provide instructions and technical support to the patient during online consultation as well as clinical visit. The patient is required to keep a record of the at-home treatment, and consult with the therapist during regular check-in. Through the repeated refinement of the virtual dream scenarios, patient achieves cognitive correction of their problem. An example of a possible dream scenario is displayed on FIG. 5. Each patient is assessed at the end of the planned computer-augmented imagery rehearsal therapy using standardized assessment tests.

### Embodiment 2

### Pain Management

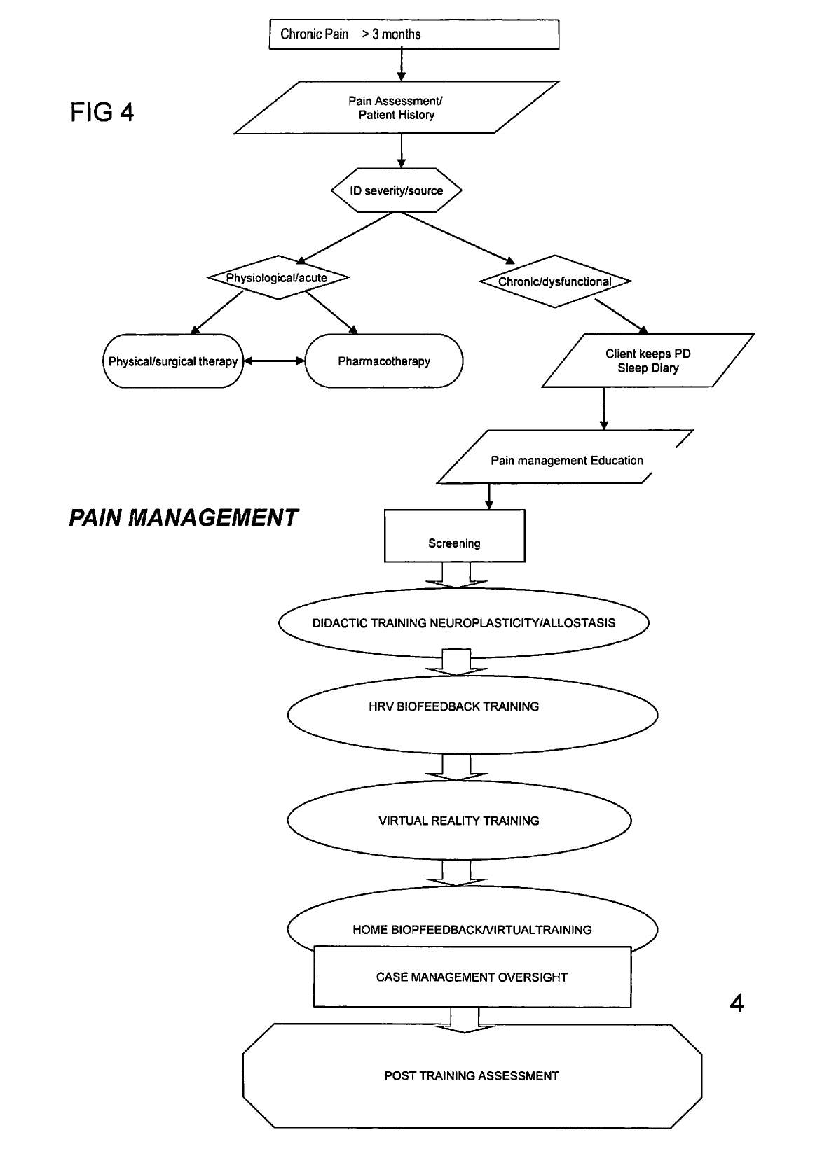

Method of treating chronic pain using computer-augmented imagery rehearsal therapy closely mirrors the treatment of sleep disorder. The procedure is illustrated in FIG. 4. The therapist first establishes Practitioner-Patient Relationship via patient interview. The patient is assessed using standard assessments, such as imaging test. Based on patient's history and assessment results, the therapist identifies the severity and diagnoses the pain. For acute pain or pains due to a diagnosed physiological dysfunction, the therapist may elect routine treatment procedures including but not limited to physical therapy, surgical corrections and/or pharmacotherapy. For patients with chronic/dysfunctional pain, the therapist may precede with computer-augmented image rehearsal therapy alone or in combination with routine treatment. Patients are educated on how pain develops based on neuophysiological/physiological models. They are introduced to the theory of neuroplasticity and allostasis and taught the role of automatic (biofeedback) regulation training and Virtual Reality training in relation to coping with pain. The patient is then screened for suitability/commitment for computer-augmented imagery rehearsal therapy. Trainee expectation and trainer's role are discussed. Automatic regulation training (ART) is provided to the patients elected for computer-augmented imagery rehearsal therapy. They are provided with hands on experience on using the physiological monitor(s) and are taught on how biofeedback impacts allostasis and neuroplasticity. Patients have opportunity to practice and familiarize themselves with the physiological monitor(s) under supervision of the trainer. Their baseline metrics are established.

Following the automatic regulation training, patients undergo virtual reality training (VRT). The trainer educates the patients about virtual reality system, explains how it works. Patients have hands on experience with the immersive environment, animation, and undergo self-paced tutorials. Patients are then given the opportunity to create his/her own avatar (virtual personality), and create their own relaxation storyboard using imageries and tools provided by the augmented reality system. The scripts are then powered into machinima (the virtual environment) and patients are allowed to practice within the virtual environment.

Following ART and VRT trainings, qualified patients are provided with equipments supporting the augmented image rehearsal treatment, and proceed to perform therapeutic training at home. Patients must complete at home trainings according to a predetermined treatment plan, which sets out the frequency, duration and tasks for the treatment. Each treatment session starts with the ART. Patient practices biofeedback relaxation techniques to prepare for VRT learning. Their physiological parameters are measured using the physiological monitor. During the follow-up VRT session, patient gain access to the virtual environment and uses the imageries and tools provided by the virtual reality system to build personalized dream scenarios and creating their own machinima. Trainer provides the patient with instructions and technical support via online consultation as well as clinical consultation. The patients are required to kept a record of their at home treatment sessions and can consult with their therapist during regular check-in. Through the refinement of the virtual scenarios, patient achieves cognitive correction. Each patient is assessed at the end of the planned computer-augmented imagery rehearsal treatment using standardized assessment tests.

### REFERENCES

1. Pimentel, K. and Teixeira, K. 1993, Virtual Reality: through the new looking glass. Intel/Windcrest/McGraw-Hill, Inc. New York).
2. R. Azuma, A Survey of Augmented Reality Presence: Teleoperators and Virtual Environments, pp. 355-385, August 1997.
3. P. Milgram and A. F. Kishino, Taxonomy of Mixed Reality Visual Displays IEICE Transactions on Information and Systems, E77-D(12), pp. 1321-1329, 1994.
4. Addison, R. (1995). Detour: brain deconstruction ahead. In: Satava, R. M., Morgan, K., Sieburg, H. B., Mattheus, R., & Christensen, J. P. Interactive technology and the new paradigm for healthcare. Pp. 1-3. IOS Press, Amsterdam, Oxford, Washington, D.C. American Psychiatric Association (1994). Diagnostic and statistical manual of mental disorders: Fourth Edition. DSM-IV. Washington,
5. D. C. Carlin, A. S., Hoffman, H. G., & Weghorst, S. (1997). Virtual reality and tactile augmentation in the treatment of spider phobia: a case report. Behavior Research Therapy, 35(2): 153-58.
6. Hodges, L. F., Rothbaum, B. O., Kooper, R., Opdyke, D., Meyer, T., North, M., de Graaff, J. J., and Williford, J. (1995). Virtual environment for treating the fear of heights. IEEE Computer 28, 7, pp. 27-34.
7. Kooper, R. (1994). Virtually present: treatment of acrophobia by using virtual reality graded exposure. Master Thesis in Computer Science at the Technical University of Delft, Netherlands.
8. Lamson, R. (1989). The effects of a manual-guided cognitive intervention program upon substance abusers. Unpublished dissertation. University of Southern California.
9. Lamson, R. (1993). The effects of virtual reality immersion on anxiety disorders. Kaiser Foundation Research Institute.
10. Lamson, R. (1994). Virtual therapy of anxiety disorders: application: VR in psychotherapy. CyberEdge Journal, Issue #20, Vol. 4, No. 2. Sausalito, Calif.
11. Lamson, R. and Meisner, M. (1994). The effects of virtual reality immersion in the treatment of anxiety, panic, and phobia of heights. Proceedings for Virtual Reality and Persons with Disabilities, pp. 63-68. Second Annual International Conference, Center on Disabilities, California State University, Northridge.
12. Lamson, R. and Meisner, M. (1995). Clinical application of virtual therapy to psychiatric disorders: theory research, practice. Pre-Conference Workshop, Medicine Meets Virtual Reality #4.
13. Pimentel, K. and Teixeira, K. (1993). Virtual Reality: through the new looking glass. Intel/Windcrest/McGraw-Hill, Inc. New York. Rothbaum, B. O., Hodges, L. F., Kooper, R., Opdyke, D., Williford, J., and North, M. (1995a). Virtual reality graded exposure in the treatment of acrophobia: a case study. Behavior Therapy, Vol. 26, No. 3, pp. 547-554. Rothbaum.
14. B. O., Hodges, L. F., Kooper, R., Opdyke, D., Williford, J., and North, M. (1995b). Effectiveness of computer-generated (virtual Reality) graded exposure in the treatment of acrophobia. American Journal of Psychiatry, Vol. 152, No. 4, pp. 626-628.
15. Foa, E., Rothbaum, B., & Furr, J. (2003). Augmenting exposure therapy with other CBT procedures. Psychiatric Annals, 33(1), 47-56.
16. Williford, J. S., Hodges, L. F., North, M. M, North, S. (1993). Relative effectiveness of virtual environment desensitization and imaginal desensitization in the treatment of acrophobia. Proceedings Graphics Interface, 162, Toronto.
17. Krakow, et al. Imagery Rehearsal Therapy for Chronic Nightmares in Sexual Assault Survivors with PTSD. JAMA. 2001; 286:537-545
18. Moore, Krakow, “Imagery Rehearsal Therapy for Acute PTSD Among Combat Soldiers in IRAQ” AM J Psychiatry 164:683-684 April 2007
19. McEwen B S. (2007) Physiology and neurobiology of stress and adaptation: central role of the brain. Physiol Rev 87:873-904.
20. McEwen B S. (2000) Allostasis and allostatic load: implications for neuropsychopharmacology. Neuropsychopharmacology 22:108-124.

What is claimed is:

1. A method for performing computer augmented Neuro-Physiological self-regulation training comprising:
(a) conducting autonomic regulation training (ART) using biofeedback responses of a subject; and
(b) conducting virtual reality training (VRT) in an interactive immersive virtual environment; and
(c) repeat step (a) and (b) to achieve therapeutic learning.

2. A method of claim 1, wherein said automatic regulation training comprising:
(a) monitoring physiological parameters of said subject;
(b) practicing relaxation techniques using biofeedback responses of said physiological parameters; and
(c) achieving a calibrated relaxation for optimal regulation state in the subject for learning.

3. A method of claim 2, wherein said physiological parameter is selected from the group consisting of heart rate, temperature, blood pressure and respiration.

4. A method of claim 1, wherein said virtual reality training comprising:
(a) accessing a machinima using tools of a virtual reality system;
(b) creating customized avatar of said subject;
(c) uploading said avatar into said machinima;
(d) creating virtual scenario using said tools of said virtual reality system; and
(e) viewing said virtual scenario.

5. The method of claim 4, further comprising practicing step (a) through (e) during a deregulation event.

6. The method of claim 5, wherein said deregulation event is a nightmare, an acute pain episode, a rage or a panic attack.

7. A method of claim 1, wherein said Neuro-Physiological self-regulation training is practiced routinely.

8. The method of claim 1, further comprising the steps of
(a) assessing functional impact of autonomic regulation on said subject's symptoms;
(b) educating the subject about the reciprocal relationship between said symptoms and autonomic regulation;
(c) training the subject on how to conduct ART and VRT, and
(d) practicing ART/VRT.

9. A method for treating sympathetic arousal subsequent to nightmare comprising:
(a) achieving a relaxed state calibrated by ART in a said subject;
(b) accessing a machinima using a virtual reality system;
(c) creating an avatar of said subject in said machinima;
(d) developing a script for dream scenario;
(d) creating said dream scenario in said machinima;
(e) reinforcing dream scenario in said machinima through repetitive practice of said dream scenario; and
(f) practicing said dream scenario when awakened by nightmare to achieve control over sympathetic arousal.

10. A method for treating acute pain episode, comprising:
(a) achieving a relaxed state calibrated by ART in a subject;
(b) accessing a machinima using a virtual reality system;
(c) creating an avatar of said subject;
(d) developing a script for a pleasurable scenario;
(e) creating said pleasurable scenario in said machinima;
(d) reinforcing said pleasurable scenario through repetitive practice of said pleasurable scenario; and
(e) applying said pleasurable scenario during breakthrough acute pain episode.
